(19) United States
(12) Patent Application Publication
(10) Pub. No.: US 2025/0152617 A1
(43) Pub. Date: May 15, 2025

(54) PHARMACEUTICAL COMPOSITION CONTAINING STIMULANT FOR PWAR5 EXPRESSION AS ACTIVE INGREDIENT FOR PREVENTING OR TREATING INFECTIOUS DISEASES DUE TO HEPATITIS C VIRUS

(71) Applicant: INDUSTRIAL COOPERATION FOUNDATION CHONBUK NATIONAL UNIVERSITY, Jeollabuk-do (KR)

(72) Inventors: Sang Min KANG, Jeollabuk-do (KR); Hee Jeong HAN, Jeollabuk-do (KR); Dong Seob TARK, Jeollabuk-do (KR); Gun Hee LEE, Jeollabuk-do (KR)

(21) Appl. No.: 18/837,408

(22) PCT Filed: Sep. 19, 2022
(86) PCT No.: PCT/KR2022/013947

§ 371 Date: Aug. 9, 2024

(30) Foreign Application Priority Data
Feb. 9, 2022 (KR) 10-2022-0016939

(51) Int. Cl. A61K 31/7105; A61P 31/14

(52) U.S. Cl. CPC A61K 31/7105; A61P 31/14

(57) ABSTRACT

The present invention relates to a pharmaceutical composition and the like containing a stimulant for PWAR5 expression as an active ingredient for preventing or treating infectious diseases due to hepatitis C virus, the present invention allowing the PWAR5 level to be sustained at a high level inside the cell to effectively inhibit proliferation of HCV.

### TECHNICAL FIELD

The present disclosure relates to a pharmaceutical composition and the like containing a stimulant for PWAR5 expression as an active ingredient for preventing or treating infectious diseases due to hepatitis C virus. The present disclosure was performed with the support of the National Institute of Health, Korea Disease Control and Prevention Agency, Academic Research Service Projects “Study on Blocking Hepatocarcinogenesis Related to HCV Infection Using Non-coding RNA Regulation Technology” (2020ER510102) and “Study on the Possibility of Drug for Blocking HCV-Induced Hepatocarcinogenesis Based on Non-coding RNA Platform” (2022ER180200). In addition, the present disclosure was performed with the support of the National Research Foundation of Korea, Science and Engineering Research Institute Support Project “Study on Development and Utilization of Rapid and Customized Genetic Engineering-Based Technology for National Disaster Disease Control” (2017R1A6A1A03015876).

### BACKGROUND ART

Since hepatitis C virus (HCV) was first found in 1989 as the main cause of post-transfusion hepatitis, the HCV has emerged worldwide as a major cause of liver disease mortality and liver transplantation due to chronic hepatitis, cirrhosis, and hepatocellular carcinoma. Although a screening test for HCV in blood transfusions has been conducted to block a major cause of infection, HCV infection through invasive procedures and the like still occurs, and there is still no vaccine capable of effectively preventing HCV. The HCV is the cause of approximately 10 to 15% of patients with liver cirrhosis and liver cancer in Korea, which is the second most important causative factor after hepatitis B virus.

Interferon monotherapy was attempted as a therapeutic agent for HCV infection in the 1990s, but the HCV was an incurable disease with a treatment success rate of only 10%. In the 2000s, the treatment success rate was increased to about 50% with combined therapy of peginterferon alpha and ribavirin, but the side effects of the drug were severe, making actual treatment difficult.

In 2014, due to the development of direct antiviral agents (DAAs), a specific therapeutic agent for HCV, a treatment success rate of over 90% is achieved when oral drugs were taken for 3 to 6 months. However, current DAA drugs are very expensive, which is a barrier to practical treatment for many patients. In addition, the HCV is an RNA virus that frequently mutates, and DAAs, which are synthetic compound therapeutic agents, are therapeutic agents that are vulnerable to variants. For this reason, there is a need to develop novel therapeutic agents that are purchased at an affordable price and may respond when variants occur.

### DISCLOSURE OF THE INVENTION

### Technical Goals

An aspect to be achieved by the present disclosure is to provide a pharmaceutical composition containing a stimulant for PWAR5 expression as an active ingredient for preventing or treating infectious diseases due to HCV, by confirming that the proliferation of HCV is reduced in PWAR5 overexpressing cells.

In addition, the present inventors have confirmed that changes in the expression level of PWAR5 are specifically associated with HCV proliferation, and thus an aspect of the present disclosure is to provide a method for providing information for differentially diagnosing patients with liver cancer caused by HCV infection among the patients with liver cancer.

However, technical goals to be achieved are not limited to those described above, and other goals not mentioned above are clearly understood by one of ordinary skill in the art from the following description.

### Technical Solutions

In order to solve the aspect, the present disclosure provides a pharmaceutical composition containing a stimulant for Prader Willi/Angelman Region RNA 5 (PWAR5) expression as an active ingredient for preventing or treating infectious diseases due to hepatitis C virus (HCV).

As one embodiment of the present disclosure, the infectious disease due to HCV may be at least one disease selected from the group consisting of hepatitis, liver fibrosis, cirrhosis, and liver cancer caused by HCV infection.

As another embodiment of the present disclosure, the stimulant for PWAR5 expression may include at least one selected from the group consisting of a polynucleotide containing a PWAR5 transcript, a complementary polynucleotide thereof, and a vector capable of expressing the same. The polynucleotide containing the PWAR5 may include modifications such as capping or tailing to maintain high stability in vivo.

In the present disclosure, a nucleotide sequence of a PWAR5 gene consists of a nucleotide sequence represented by SEQ ID NO: 1, and the PWAR5 transcript may be an RNA strand transcribed from the PWAR5 gene.

As yet another embodiment of the present disclosure, the stimulant for PWAR5 expression may inhibit the proliferation of HCV in vivo.

In addition, the present disclosure provides a method for providing information for differentially diagnosing liver cancer caused by HCV infection, including the following steps:
(1) isolating total RNA from liver cancer cells isolated from a liver cancer patient;
(2) measuring a level of PWAR5 in the total RNA;
(3) comparing the level of PWAR5 with an expression level of PWAR5 in normal hepatocytes; and
(4) determining that the patient has liver cancer caused by HCV infection when the expression level of PWAR5 in the liver cancer cells of the liver cancer patient is lower than that of normal hepatocytes.

As an embodiment of the present disclosure, the expression level of PWAR5 may be measured by one or more methods selected from the group consisting of RT-PCR, competitive RT-PCR, real-time RT-PCR, RNase protection assay (RPA), Northern blotting, DNA chip, and Fluorescence-activated cell sorting (FACS).

As another embodiment of the present disclosure, in step 4, if the expression level of PWAR5 in liver cancer cells is at least 50% lower than that of normal hepatocytes, the patient in step 1 may be determined to be a liver cancer patient caused by HCV infection.

In addition, the present disclosure provides a method for preventing or treating infectious diseases due to HCV including administering a stimulant for PWAR5 expression to a subject.

As one embodiment of the present disclosure, the method for treating infectious diseases due to HCV may further include measuring a level of a HCV gene in a biological sample isolated from the subject. The measuring of the level of the HCV gene in the sample may be performed before and/or after the administering step, and when the levels of the HCV gene are measured before and after the administration, the method may further include comparing the levels of the gene before and after the administration.

In addition, the present disclosure provides a use of a stimulant for PWAR5 expression for the preparation of a drug for preventing or treating infectious diseases due to HCV.

### Effects of the Invention

According to the present disclosure, it is possible to provide PWAR5 as a novel target for the prevention and treatment of infectious diseases due to hepatitis C virus, and to effectively inhibit proliferation of HCV by providing the present disclosure to maintain the PWAR5 at a high level within cells.

### BRIEF DESCRIPTION OF DRAWINGS

FIG. 1 is a graph comparing the expression levels of PWAR5 in a liver cancer tissue and a non-liver cancer tissue among liver tissues of a HCV-positive liver cancer patient.

FIG. 2 is a graph comparing the expression levels of PWAR5 in liver cancer cells and normal hepatocytes.

FIG. 3 is a graph confirming that the expression level of PWAR5 decreases with proliferation of HCV in Huh7.5 cells as a liver cancer cell line.

FIG. 4 is a graph confirming that the expression level of PWAR5 decreases with proliferation of HCV in PHH cells as a normal hepatocyte line.

FIG. 5 is a graph confirming the proliferation of HBV and changes in the expression level of PWAR5 in HepG2-NTCP cells as a liver cancer cell line.

FIG. 6 is a graph confirming that proliferation of HCV is reduced as PWAR5 expression increases in PWAR5 overexpressing cells.

FIG. 7 is a graph confirming that the proliferation ability of HCV is reduced in PWAR5 stable cells.

FIG. 8 is a diagram showing results of confirming luciferase activity 48 hours after infecting A Huh7.5 cell line with HCVpp or VSVpp.

FIG. 9 is a diagram confirming a decrease in translation by HCV IRES according to PWAR5 expression in a Huh7.5 cell line.

FIG. 10 is a diagram confirming that the expression of negative strand RNA of HCV is reduced according to PWAR5 expression in a Huh7.5 cell line.

### BEST MODE FOR CARRYING OUT THE INVENTION

The present disclosure was completed by confirming that the expression level of PWAR5 was negatively correlated with HCV proliferation, and that an increase in the expression level of PWAR5 may effectively inhibit HCV proliferation.

More specifically, the present inventors confirmed that the expression level of lncRNA, PWAR5 in liver cancer tissue among liver tissues of patients with HCV-mediated liver cancer was significantly lower than that in non-liver cancer tissue (see Example 1).

In addition, the present inventors confirmed that PWAR5 was highly expressed in normal hepatocytes, but the expression there of was reduced in liver cancer cells by measuring the expression level of PWAR5 in normal hepatocytes and liver cancer cells, and presumed that the decrease in PWAR5 expression would be related to the progression of normal hepatocytes into cancer cells (see Example 2).

Accordingly, the present inventors infected normal hepatocytes and liver cancer cells with HCV and confirmed an HCV RNA level and a PWAR5 expression level to confirm a correlation between the PWAR5 expression level and the HCV proliferation through a specific experiment and thus it can be seen that the PWAR5 expression level changes specifically in HCV infection (see Example 3). The results were verified by the fact that no significant change in PWAR5 expression level was observed in HepG2 cells infected with hepatitis B virus (HBV) (see Example 4).

Furthermore, the present inventors confirmed the HCV RNA expression level in cells overexpressing PWAR5 by transforming a PWAR5 gene to verify the effect of PWAR5 as a new target for prevention or treatment of HCV and as a result, confirmed that as the expression level of PWAR5 in cells increased, the expression levels of HCV RNA and viral protein decreased. Thus, it can be seen that a decrease in PWAR5 expression is not simply a phenomenon resulting from HCV proliferation, but rather a necessary condition for HCV proliferation (see Example 5). The results were re-verified by confirming that the proliferation ability of HCV was reduced in PWAR5 stable cells (see Example 6).

Next, the present inventors confirmed the effect of PWAR5 on the life cycle of HCV. Specifically, in a hepatocyte line, PWAR5 did not affect HCV entry, but affected a HCV IRES operation to inhibit protein translation and significantly reduced the expression of negative strand RNA for HCV proliferation (see Example 7).

Accordingly, the present inventors can provide PWAR5 as a new target for preventing or treating infectious diseases due to HCV and provide a pharmaceutical composition containing a stimulant for PWAR5 expression as an active ingredient for preventing or treating infectious diseases due to HCV.

Meanwhile, the present inventors can provide a method for differentially diagnosing liver cancer patients caused by HCV infection from liver cancer patients by measuring the level of PWAR5 of which the expression level is specifically reduced in particularly HCV infection among hepatitis viruses.

Prader Willi/Angelman Region RNA 5 (PWAR5) is long non-coding RNA (lncRNA). The lncRNA is known to interact with other DNA, RNA, or proteins to regulate the structure or function thereof and the PWAR5 is known to be related to the occurrence of brain tumors, but a relationship with HCV replication has not yet been found.

As used herein, the “stimulant for PWAR5 expression” is not limited to anything that may increase the level of PWAR5 in hepatocytes, and non-limiting examples thereof may include a polynucleotide consisting of or including a nucleotide sequence of PWAR5, a complementary polynucleotide thereof, or a vector capable of expressing the same.

As used herein, the “nucleotide” or “polynucleotide” refers to deoxyribonucleotide or ribonucleotide that exists in a single-stranded or double-stranded form, and includes analogues of natural (poly) nucleotides unless specifically stated otherwise.

In this specification, transfection or transduction means introducing foreign DNA or RNA into a cell. The transfection may be performed by many methods known in the art, such as calcium phosphate-DNA co-precipitation, DEAE-dextran-mediated transfection, polybrene-mediated transfection, electroporation, microinjection, liposome fusion, lipofectamine, and protoplast fusion. The transfection means transferring a gene into a cell using virus or viral vector particles by means of infection. In this specification, the transfection and the transduction may be used interchangeably, but are desirably interpreted as transformation of foreign gene transfer into a host cell in a broad sense, and a cell into which a foreign gene has been introduced through transfection or transduction is called a transformant.

Meanwhile, the transfection may be classified into stable transfection if the transfected foreign gene is inserted into the chromosomal DNA of a host cell or is in a form that can replicate independently, and transient transfection if not.

In the present disclosure, the stimulant for PWAR5 expression may be introduced into cells using various transformation techniques, such as complexes of DNA and DEAE-dextran, complexes of DNA and nuclear proteins, and complexes of DNA and lipids, and to this end, the stimulant for PWAR5 expression may be included in a carrier that enables efficient introduction into the cell. The carrier is desirably a vector, and both a viral vector and a non-viral vector can be used. For example, the viral vector may be used with lentivirus, retrovirus, adenovirus, herpes virus, and avipox virus vectors, and the like, desirably a lentivirus vector, but is not limited thereto. The lentivirus is a type of retrovirus that may infect both division cells and non-division cells due to the nucleophilicity of a pre-integration complex (viral “shell”) that allows active entry into a nucleopore or a complete nuclear envelope.

The stimulant for PWAR5 expression of the present disclosure may be isolated or prepared using standard molecular biology techniques, for example, a chemical synthesis method or a recombinant method, or may use a commercially available product.

As used herein, the ‘prevention’ means all actions that delay the onset of infectious diseases due to HCV by inhibiting the proliferation of HCV through administration of the composition, and the ‘treatment’ means all actions that improve or beneficially change the symptoms of the disease through administration of the composition.

The pharmaceutical composition of the present disclosure may further include a pharmaceutically acceptable carrier in addition to the stimulant for PWAR5 expression, and may be formulated with the carrier. As used herein, the term “pharmaceutically acceptable carrier” may refer to a carrier or a diluent which does not inhibit biological activity and properties of a compound to be administered without stimulating organisms. In the composition formulated with a liquid solution, the pharmaceutically acceptable carrier is suitable for sterilization and living bodies and may use saline, sterilized water, ringer's solution, buffered saline, albumin injection solution, dextrose solution, maltodextrin solution, glycerol, ethanol, and a mixture of at least one of these ingredients, and if necessary, may add other general additives such as antioxidants, buffers, bacteriostatic agents, and the like. In addition, the pharmaceutical composition may be formulated in injectable formulations such as an aqueous solution, a suspension, and an emulsion, pills, capsules, granules, or tablets by further adding a diluent, a dispersant, a surfactant, a binder, and a lubricant.

The pharmaceutical composition of the present disclosure can also be applied to any formulation containing the pharmaceutical composition as an active ingredient, and may be prepared as an oral or parenteral formulation. The pharmaceutical formulation of the present disclosure includes a form suitable for oral, rectal, nasal, topical (including buccal and sublingual), subcutaneous, or parenteral (including intramuscular, subcutaneous, and intravenous) administration or for administration by inhalation or insufflation.

Formulations for oral administration containing the composition of the present disclosure as an active ingredient may be prepared, for example, as tablets, troches, lozenges, aqueous or oily suspensions, prepared powders or granules, emulsions, hard or soft capsules, syrups or elixirs. For preparation into formulations such as tablets and capsules, the composition may contain a binder such as lactose, saccharose, sorbitol, mannitol, starch, amylopectin, cellulose or gelatin, an excipient such as dicalcium phosphate, a disintegrate such as corn starch or sweet potato starch, a lubricant such as magnesium stearate, calcium stearate, sodium stearyl fumarate or polyethylene glycol wax. In the case of capsule formation, the composition may further contain a liquid carrier such as fatty oil in addition to the aforementioned materials.

The formulations for parenteral administration containing the composition of the present disclosure as an active ingredient may be prepared in an injectable form, such as subcutaneous injection, intravenous injection, or intramuscular injection. For the preparation of injectable formulation, the composition of the present disclosure may be mixed in water with a stabilizer or buffer to be prepared as a solution or suspension, and may be formulated for unit dosage of ampoules or vials. For injection as a suppository, the composition may be formulated as a rectal composition, such as a suppository or cure enema containing a conventional suppository base, such as cocoa butter or other glycerides.

Hereinafter, embodiments will be described in detail with reference to the accompanying drawings. However, since various modifications may be made to embodiments, the scope of the present disclosure is not limited or restricted by these embodiments. It should be understood that all modifications, equivalents and substitutes for embodiments are included in the scope of the present disclosure.

The terms used in embodiments are used for the purpose of description only, and should not be construed to be limited. A singular expression includes a plural expression unless the context clearly indicates otherwise. In the present disclosure, it should be understood that term “comprising” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which embodiments pertain. Terms which are defined in a generally used dictionary should be interpreted to have the same meaning as the meaning in the context of the related art, and are not interpreted as ideal or excessively formal meanings unless otherwise defined in the present disclosure.

In addition, in the description with reference to the accompanying drawings, like components designate like reference numerals regardless of reference numerals and a duplicated description thereof will be omitted. In describing the embodiments, a detailed description of related known technologies will be omitted if it is determined that they unnecessarily make the gist of the embodiments unclear.

### Experimental Methods

### 1. Cell Culture

Huh7.5 was incubated in a Dulbecco's modified Eagle's medium (DMEM; Gibco) containing 10% fetal bovine serum (FBS: Gibco), 100 U/mL penicillin, 100 mg/mL streptomycin (Gibco), and a non-essential amino acid solution (NEAA). Huh7 and HepG2 were incubated in a Dulbecco's modified Eagle's medium (DMEM: Gibco) containing 10% fetal bovine serum (FBS: Gibco), 100 U/mL penicillin, and 100 mg/mL streptomycin (Gibco). Human hepatocytes were purchased as Gentest™ Plateable Human CryoHepatocytes from Corning. Cryopreserved cells were completely thawed in a 37° C. water bath for 2 minutes or more. The hepatocytes were added in a Gibco Hepatocyte Thaw Medium and centrifuged at 150×g for 5 minutes at room temperature. The precipitated cells were added in a William's E Medium and centrifuged at 150×g for 5 minutes. The cells were incubated on a type I collagen-coated plate and incubated in a humidified cell incubator at 37° C. and 5% CO₂/95% air for one day.

### 2. Plasmid Assembly and Transfection

A cDNA encoding a PWAR5 transcript represented by SEQ ID NO: 1 was synthesized and cloned into BamHI/EcoRI sites of a pcDNA3.1 vector (genscript). In addition, cDNA encoding the PWAR5 transcript was cloned into BamHI/EcoRI sites of a pLV-EFla-IRES-hygro vector. The cells were transformed with pcDNA3.1 and pcDNA3.1-PWAR5 using lipofectamine2000).

### 3. Production of PWAR5 Stable Cells

To produce lentivirus inserted with PWAR5, HEK293T was incubated in a 100 mm dish. Plasmids of PMD2.G, psPAX2-Gal/Pol, and pLV-EF1A-PWAR were mixed with 4 μg each of lipofectamin3000 and treated to the cells. The supernatant was harvested 48 hours after transfection and centrifuged at 150×g for 5 minutes to remove cell debris. A Lenti-X™ Concentrator (TaKaRa) and the supernatant were mixed in a 1:3 ratio and reacted overnight at 4° C. The supernatant mixed with the concentrator was centrifuged at 1500×g for 45 minutes to be removed, and then the remaining pellet was suspended in a culture medium without antibiotics or FBS. Huh7.5 was inoculated at 8×10⁵ in 6 wells, and after 16 hours, was infected with a mixture of 8 μg/ml polybrene and lentivirus. After 48 hours, the medium was replaced with a medium containing 400 μg/ml hygromycin to select cells expressing PWAR5.

### 4. Production and Infection of Cell Culture-Derived Hepatitis C Virus (HCVcc)

pJcl DNA reacted with a restriction enzyme (200 U Mlu I) at 37° C. for 1 hour for degradation. In vitro RNA synthesis was performed using a T7 RiboMAX™ Express Large Scale RNA Production System (Promega), and the synthesized RNA was purified using a TRIzol™ LS reagent. Next, Huh7.5 cells were mixed with 400 μl cytomix buffer (360 μl cytomix+20 μl GSH+20 μl ATP) and Jcl RNA, and electroporated using Gene Pulser Xcell Electroporation Systems (bio-rad) (270 V and 950 uF). The transfected cells were immediately transferred to a T175 culture flask filled with a culture medium and incubated at 37° C. for 72 hours. The culture medium containing hepatitis C virus was collected and centrifuged at 1,000 g for 20 minutes to harvest the virus. Hepatitis C virus titers were measured using an immunofluorescence assay.

Huh7.5 was inoculated at 4×10⁵ cells in a 12-well plate, and after 16 hours, the cells were infected with 0.05 moi of hepatitis C virus. Normal hepatocytes were inoculated at 4×10⁵ cells in a type I collagen-coated 12-well plate. The cells were then infected four times consecutively with 1 moi hepatitis C virus.

5. Quantitative Real-Time Polymerase Chain Reaction (qRT-PCT)

Total RNA from cells was purified using a TRIzol reagent, and qRT-PCR was performed according to the manufacturer's protocol. Specifically, 500 ng RNA was synthesized to cDNA using a reverse transcription reagent kit (Invitrogen). 10 μL of SYBR® Green Master Mix (bio-rad), 5 pmol each of forward and reverse primers, and 1 μL of cDNA were mixed and then qRT-PCR was performed using Quantstudio 3 Real-Time PCR (TheromoFisher). The sequences of PCR primers were shown in Table 1 below.

TABLE 1

| | | SEQ | |
|---|---|---|---|
| | | ID | |
| Gene | Primer | NO: | Sequence (5′->3′) |
| PWAR5 | forward | 2 | TGATGTGGGTGTTGATAC |
| | reverse | 3 | ATCAAGAACGGAAACTCA |
| qJFH1 | forward | 4 | TTAGTATGAGTGTCGTACAGCCTCCAG |
| | reverse | 5 | GGCATAGAGTGGGTTTATCCAAGAAAGG |
| HBV | forward | 6 | TCACCAGCACCATGCAAC |
| | reverse | 7 | AAGCCACCCAAGGCACAG |
| GAPDH | forward | 8 | ACAGTCAGCCGCATCTTCTT |
| | reverse | 9 | ACGACCAAATCCGTTGACTC |

### 6. Western Blot Assay

Liver cancer cells and normal hepatocytes were added with 0.3 ml of RIPA lysis buffer, and then centrifuged at 13,000 rpm and 4° C. for 5 minutes to obtain the supernatant. The supernatant was mixed with a protein sample buffer at 1:3 and then heated at 100° C. for 5 minutes. Protein samples were loaded onto an SDS-PAGE gel, transferred to a nitrocellulose (NC) membrane, and blocked with 5% skim milk for 1 hour. Primary antibodies HCV NS5A, core, and GAPHD were diluted in a TBS-T buffer containing 1% skim milk and reacted at 4° C. The next day, the membrane was washed six times for 5 minutes each with a TBS-T buffer, and a secondary antibody was diluted in a TBS-T buffer and treated to the membrane. After incubation for 1 hour at room temperature, the membrane was washed six times with a TBS-T buffer for 5 minutes again, treated with an ECL substrate, and then protein development was performed using an X-ray film.

### 7. HBV Infection

HepG2-NTCP cells were inoculated at 8×10⁵ in a 6-well plate. The medium was replaced with DMEM containing 1×NEAA, 3% FBS, 4% PEG8000, and 2% DMSO, and infected with 1000 Geq HBV. After 20 hours, the cells were washed twice with free media and added with new DMEM containing 3% FBS and 2% DMSO. The cells were treated with TRIzol 3, 5, and 7 days after infection and then RNA was purified.

### 8. Production of Pseudoparticles and Entry Assay

HEK293T cells were transfected with a transfer vector containing a firefly luciferase reporter gene, a gag-pol packing plasmid, an HCV E1E2 (genotype 2a) plasmid, or a VSV G envelope expression plasmid using lipofectamin 3000 (Thermo Fisher Scientific: L3000001). 48 hours after transfection, the supernatant containing HCVpp and VSVpp was collected and centrifuged to remove cell debris. The supernatant was concentrated using Amicon Ultra-15 to produce high concentrations of HCV pseudoparticles (HCVpp) and vesicular stomatitis virus pseudoparticles (VSVpp). Next, Huh7.5 was inoculated at 3×10⁴/well in a 96-well plate and transfected with 150 ng of pcDNA3.1-PWAR5 or pcDNA3.1-vector the next day. After 24 hours, the cells were infected with HCVpp or VSVpp. After 48 hours of infection, luciferase activity was confirmed using the Bright-Glo™ Luciferase Assay System (promega: E2610).

### 9. Luciferase Reporter Assay

Huh7.5 was incubated in a 96-well plate, and transfected with pcDNA3.1-empty plasmid or pcDNA3.1 PWAR5 plasmid after 16 hours. After 24 hours, a pRL-HL plasmid containing both HCV IRES tagged with the firefly luciferase gene and a cytomegalovirus (CMV) gene tagged with the Renilla luciferase gene was transfected. Dual-luciferase assays (promega: E2920) were performed after 18 hours of transfection.

### 10. Fluorescence In Situ Hybridization (FISH)

Probes capable of detecting Negative-strand RNA (441371-C2), Positive-strand RNA (441361-C3), and PWAR5 gene (1086561-C1) used in the experiment were prepared through ACD. Huh7.5 PWAR5 stable cells and Huh7.5 Vector stable cells were incubated at 1.5×10⁵/well on Cell Culture Slide (SPL: 30504). After 16 hours, the cells were infected with 0.1 moi of HCV, and after 4 hours, the medium was replaced with a fresh medium. After 24 and 48 hours of infection, the culture medium was removed and the cells were washed with PBS. Thereafter, the cells were immobilized by adding 10% Neutral buffered formalin (sigma: Cat. HT501128) and reacting at room temperature for 30 minutes. Thereafter, RNA was stained using RNAscope multiplex fluorescent reagent kit v2 (ACD: 323132).

### 11. RNA-Tissue Microarray (TMA)

To confirm the expression of PWAR5 in virally induced liver tissue and normal liver tissue, a Tissue Microarray Panel (Bibmax: LV8013a) was purchased. In situ Hybridization was performed in all experiments using a RBAscope 2.5 HD Duplex Reagent Kit (ACD: 322430), and referred to the usage of the product. All the tissues were stained using a PWAR5 probe (1086561-C1) and GAPDH (ACD: 414401-C2) as a positive control probe.

### EXAMPLE

### Example 1. Confirmation of Expression Levels of RWAR5 in Tumor Tissue and Non-Tumor Tissue of HCV-Infected Liver Tissues

Liver tissue samples from HCV-positive liver cancer patients were provided by the National Biobank of Korea (n=20).

Tumor tissue and non-tumor tissue were distinguished from liver tissues of 20 HCV-positive liver cancer patients, and total RNA was isolated from each region, and the expression level of lncRNA, PWAR5 was confirmed.

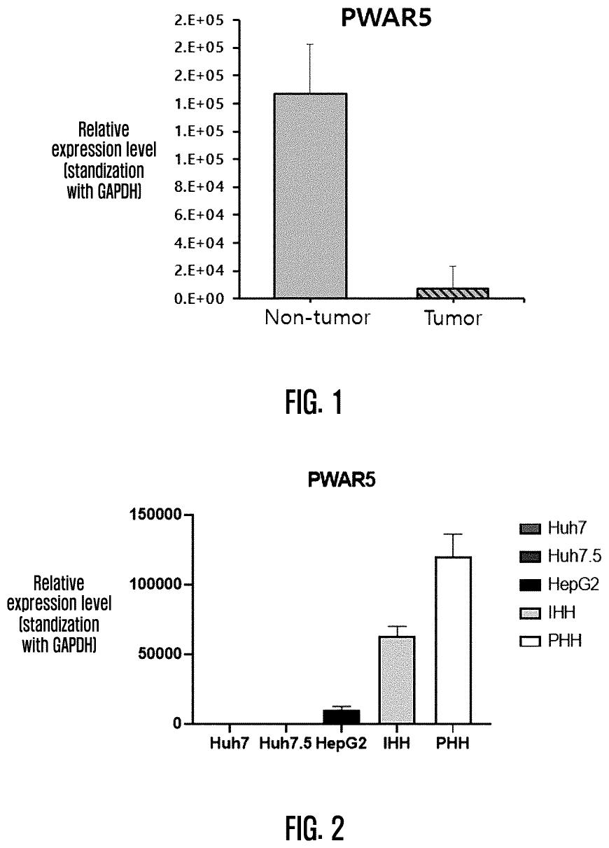

As a result, as illustrated in FIG. 1, it was confirmed that the expression level of PWAR5 in the tumor tissue was significantly lower than that in the non-tumor tissue.

### Example 2. Confirmation of Expression Levels of PWAR5 in Various Hepatocytes

The expression levels of PWAR5 were confirmed in a PHH cell line, which was primary human hepatocytes, an IHH cell line, which were immortalized human hepatocytes, and HepG2, Huh7, and Huh7.5 cell lines, which were liver cancer cells. PHH cells and IHH cells, in which HCV infection and proliferation were confirmed, Huh7 and Huh7.5 cells with high HCV infection and proliferation rates, and HepG2 cells, which were HCV infection-negative, were used. As a result, as illustrated in FIG. 2, it was confirmed that the expression level of PWAR5 in the liver cancer cells was significantly lower than that in the primary human hepatocytes and the immortalized human hepatocytes. Furthermore, it was confirmed that the Huh7 and Huh7.5 cell lines capable of HCV infection had lower expression levels of PWAR5 than the HepG2 cell line that was not infected with HCV.

### Example 3. Confirmation of Correlation Between PWAR5 Expression Level and HCV Replication

### 3-1. Confirmation of Correlation Between PWAR5 Expression and HCV Proliferation in Liver Cancer Cell Line

To confirm whether HCV infection affected the expression level of PWAR5 in hepatocytes, Huh7.5 cells were infected with HCV, and 4 days after infection, total RNA was isolated from the cells to confirm the expression level of PWAR5.

As a result, as illustrated in FIG. 3, it was confirmed that as the amount of HCV RNA increased in liver cancer cells after HCV infection, the amount of PWAR5 RNA decreased, and from the results, it was found that there was a negative correlation between HCV proliferation and the expression level of PWAR5.

### 3-2. Confirmation of Correlation Between PWAR5 Expression and HCV Proliferation in Normal Cell Line

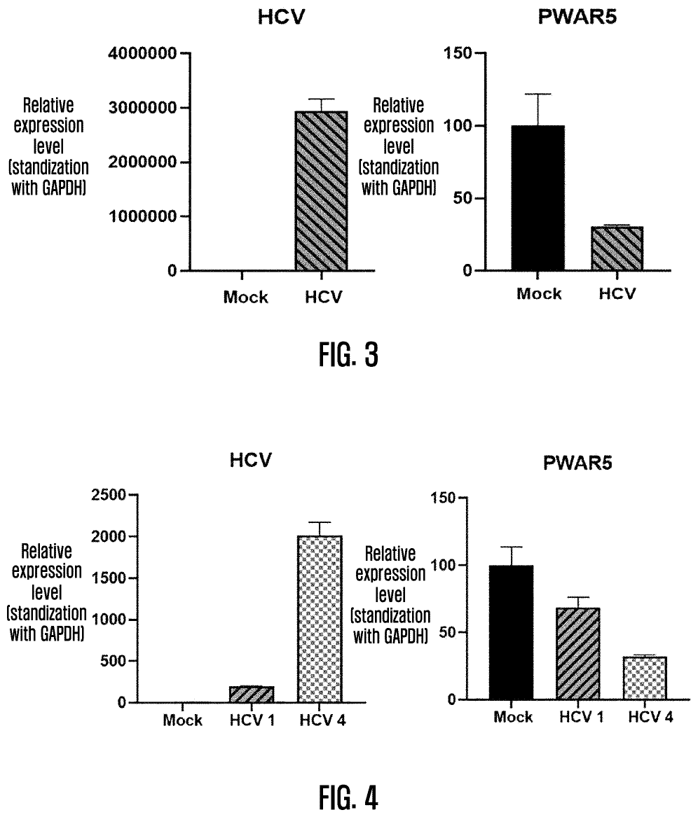

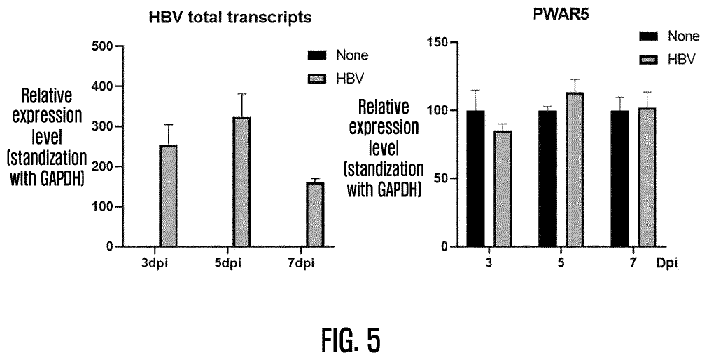

To determine whether the correlation between HCV proliferation and PWAR5 expression levels in liver cancer cells was equally applied to normal cell lines, PHH cells, which were normal hepatocytes, were infected with HCV and the expression level of PWAR5 according to HCV proliferation was determined. In the case of PHH cells, the HCV infection rate and proliferation rate were very low; and thus after infected with HCV once and four times for 7 days, the amount of HCV RNA and the expression level of PWAR5 were confirmed. As a result, as illustrated in FIG. 4, it was confirmed that as the amount of HCV RNA increased in PHH cells after HCV infection, the amount of PWAR5 RNA decreased. From the results, it can be seen that there is a negative correlation between the HCV proliferation and the expression level of PWAR5 even in normal cells, and that the expression level of PWAR5 changes specifically in HCV infection.

### Example 4. Confirmation of Relationship Between PWAR5 Expression and Hepatitis B Virus (HBV) Proliferation

Hepatitis viruses are a major cause of liver cancer. The hepatitis viruses include six virus types of A, B, C, D, E, and G, and in particular, hepatitis B and C viruses are known to be the major causes of liver cancer. Hereinafter, it was confirmed whether the PWAR5 expression level was negatively correlated with HBV proliferation. Specifically; HepG2-NTCP cells capable of HBV infection were infected with HBV, and the expression level of PWAR5 was confirmed on days 3, 5, and 7.

As a result, as illustrated in FIG. 5, it was confirmed that HBV was infected and proliferated in HepG2-NTCP cells, and there was no significant correlation between the proliferation of HBV and the expression level of PWAR5.

### Example 5. Confirmation of Decreased HCV Proliferation Upon PWAR5 Overexpression

In a phenomenon where PWAR5 expression decreased due to HCV proliferation in normal hepatocytes and liver cancer cells, it was confirmed whether the decrease in PWAR5 expression was simply a symptom due to HCV proliferation or a necessary condition for HCV proliferation. Therefore, to artificially overexpress PWAR5, Huh7.5 cells were transfected with a PWAR5 gene, and infected with HCV, and then after 48 hours, the cells were harvested to confirm the level of PWAR5 and the amount of HCV RNA in the cells.

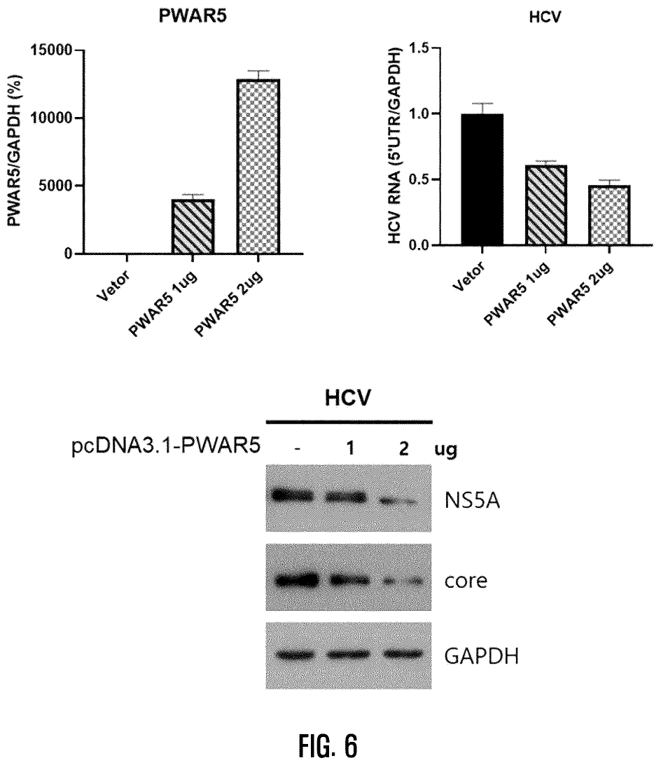

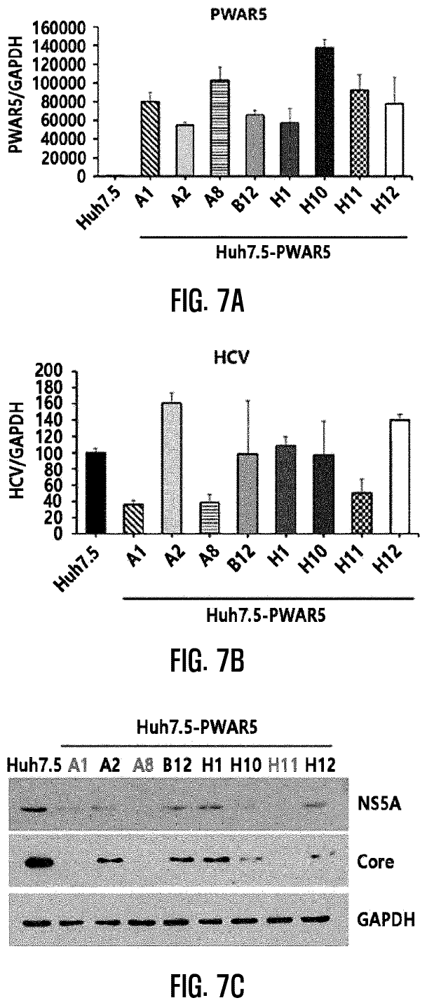

As a result, as illustrated in FIG. 6, it was confirmed that the expression level of PWAR5 increased in proportion to the amount of transfected gene, and HCV RNA expression decreased as the expression level of PWAR5 increased. This was verified by confirming a decrease in the levels of viral proteins core and NS5A.

### Example 6. Confirmation of HCV Proliferation Ability in PWAR5 Stable Cells

lncRNA was generally known to have less stability and a shorter half-life than cellular mRNA. To re-verify whether overexpression of PWAR5 may block HCV replication, PWAR5 stable cells were constructed, and the cells were sorted into single cells, and total eight types of single cells were selected. The selected eight types of single cells were infected with HCV and the proliferation ability of the virus was confirmed.

As a result, it was confirmed that the HCV RNA expression level was low in A1, A8, and H11 cells with high PWAR5 expression levels, and the expression levels of the viral proteins core and NS5A were also low compared to other cells.

### Example 7. Confirmation of PWAR5 Expression and HCV Life Cycle

### 7-1. Confirmation of Effect on HCV Entry

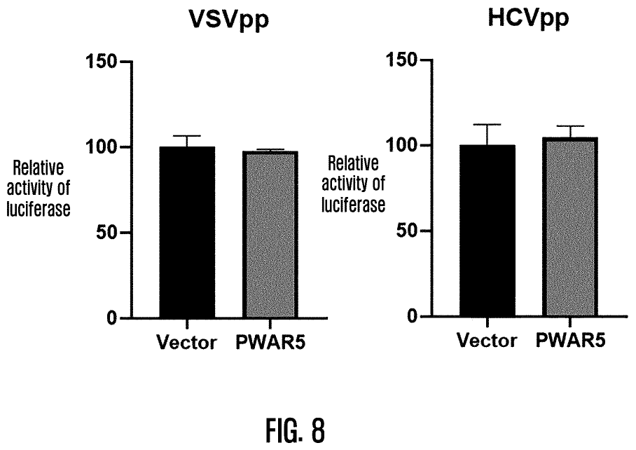

To confirm whether PWAR5 expression was involved in HCV entry, HCV pseudovirus (HCVpp) expressing E1 and E2, which were important for HCV infection, was produced. Vesicular stomatitis virus (VSVpp) was used as a comparative group. Luciferase activity was confirmed 48 hours after infection with HCVpp or VSVpp, but there was no change in a luciferase value due to the expression of PAWR5. Through this, it was confirmed that the PWAR5 expression did not affect HCV entry (FIG. 8).

### 7-2. Confirmation of Effect on HCV Translation

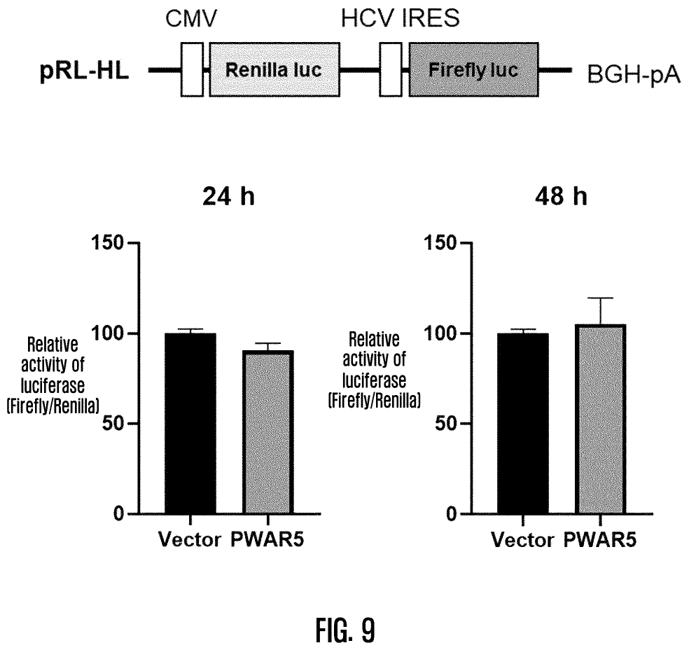

Since proliferation of HCV was regulated by translation through an internal ribosome entry site (IRES), it was investigated whether PWAR5 was involved in an HCV translation step using an IRES plasmid tagged with a luciferase gene. However, even when PWAR5 was highly expressed, luciferase activity was not significantly changed. Therefore, it was confirmed that PWAR5 did not affect IRES-dependent translation (FIG. 9).

### 7-3. Confirmation of Effect on HCV Replication

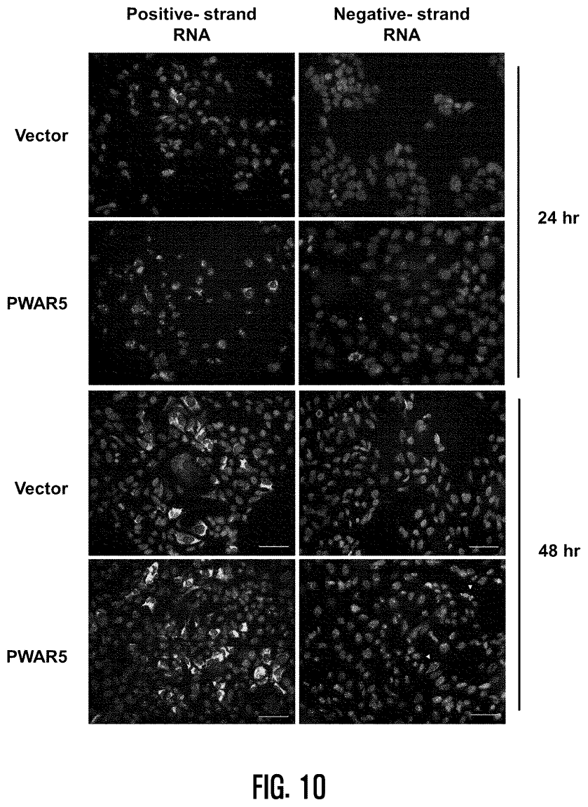

Next, to determine whether the expression of PWAR5 affected HCV replication, the expression of HCV positive-strand RNA and negative-strand RNA according to HCV infection time was confirmed. In situ hybridization assay capable of directly confirming a viral gene expressed within cells was performed. All experiments were performed by observing the expression of positive-strand RNA and negative-strand RNA of HCV using a fluorescence microscope. As a result of confirming the expression of HCV RNA 24 hours after infection, it was confirmed that the expression of RNA was low and did not show a significant difference with the expression of PWAR5. After 48 hours, there was no significant difference in the expression of positive strand RNA in cells expressing PWAR5, but the expression of negative strand RNA was significantly reduced. In addition, it was confirmed that PWAR5 stable cells were subjected to single cell selection, and most cells had high expression of PWAR5, but occasionally, cells that did not express PWAR5 were mixed therewith. Among the cells, negative stand RNA was detected only in cells that did not express PWAR5 (FIG. 10).

As described above, although the embodiments have been described by the restricted drawings, various modifications and variations can be applied on the basis of the embodiments by those skilled in the art. For example, even if the described techniques are performed in a different order from the described method, and/or components such as a system, a structure, a device, a circuit, and the like described above are coupled or combined in a different form from the described method, or replaced or substituted by other components or equivalents, an appropriate result can be achieved.

Therefore, other implementations, other embodiments, and equivalents to the appended claims fall within the scope of the claims to be described below.

1. A method for preventing or treating infectious diseases due to hepatitis C virus (HCV), comprising administering a pharmaceutical composition containing a stimulant for Prader Willi/Angelman Region RNA 5 (PWAR5) expression to a subject.

2. The method of claim 1, wherein the stimulant for PWAR5 expression comprises at least one selected from the group consisting of a polynucleotide containing PWAR5, a complementary polynucleotide thereof, and a vector capable of expressing the polynucleotide.

3. The method of claim 1, wherein the stimulant for PWAR5 expression comprises a nucleotide sequence represented by SEQ ID NO: 1.

4. The method of claim 1, wherein the composition inhibits proliferation of HCV.

5. The method of claim 1, wherein the infectious disease due to HCV is at least one disease selected from the group consisting of hepatitis, liver fibrosis, cirrhosis, and liver cancer caused by HCV infection.

6. A method for providing information for differentially diagnosing liver cancer caused by hepatitis C virus (HCV) infection, comprising:
(1) measuring the expression level of PWAR5 in liver cancer cells isolated from a liver cancer patient; and
(2) determining that the patient is a liver cancer patient caused by HCV infection when the expression level of PWAR5 is 50% or less compared to the expression level of PWAR5 in normal liver cells.

7. (canceled)

8. (canceled)

9. A composition for inhibiting proliferation of hepatitis C virus (HCV) containing a stimulant for Prader Willi/Angelman Region RNA 5 (PWAR5) expression as an active ingredient.


SEQUENCE LISTING (9 sequences)

SEQ ID NO: 1 - DNA, 3373 bp, Homo sapiens
  source 1..3373: mol_type = genomic DNA
tttccttcatgtagattgctgtgtgtgtctttttcttcataattccccttaattctgtgtgctttgaatgacactgttgttgttactgttgttttatatccattgtcacataaaactcttatcatttattcttccaggccagggactgtccttatgggtagagatacactatctgcaggaatttatttttttacgtatttttactagttgatgggaaagacattgggaaagacctgggctggatttgtgatgagctgtgtttactgagcatgatgaagtaaagctcaacgtgattactctgaagtccagccttactgtcagtctaaggactcaggatacaaatactcaaaacataaattcttagactctaaatttcacatggtttttcctatcagtacaggttaaggataatggcgtgttgtaggtttttccagtgcaatacaaggaagttggttctagatcctttagtacagttaatgttttgtagagtgtgttctatgttcactgcattggtatgtctcatctttggcatgatcttggctcaccgcaacctccgcctcttgcattcaagcacttcttgtctcagcctccagagtagctgggattacaagcatgtgccaacatgcccggctaatttttgtatgcttttgtggagacaggctttgccatgtttgccaggttggtctctaagtgatccacccgcctcagccccacaaagtgctgggattacatgcgtgagccaccgtacccagccacacctttgcttatttctactatgcatttcaagtgatcatttcaagagaataagtacttaggagctcctatgctctttttttttttaaccctgttgcttgcatcacccggttccaaccctatatgaaagtttcatgagcatgtttctgtcactgaggcagcttctccatttggcatataattgagaaattgctagtttgtggctgagacattggtttgagaggagacgtaatttatatatttagcagaatgcttaagttacatttccagagattaatttaattctacttttaattaagttaaccctttgctgtatctttacaagacgtaggtggagatttaattttgactcttagattcagttaaggtgatgaaattggcattatatagtttttgatgctgagccccacggagattaaaagatttgctaaacaccatgcaatttttgaatcacatctctaatctcacattattctgtactctattttttgcttgtatgattgatgggtctttcattatctgtgattgacattctatgagtaggtgcttttgctttgcctataagtcgttattatgaaggaggaatggtgaataagaaggtaatttagaaaagcctatattaaatataccatgaacattgaatatagcaagatcttattctctagttgttatcttagttgataaattctgtatgtgttatgtgtttgtgtatacatatgtacatattaatgatgtgggtgttgatactgtgatcacatgacaaatgactggtttatagtgaaaagctgttggggttcagagaaatgaacattcttctaaaagtgaggtagataagttattttatagttcttgcctttgagtctctgttcttctggtagaaatgttagtgtatttataacctctcagatttgtttggttctcttagtgttcttgatccaggcagttagcgatatgttggatgaattgagaaaaatgagtttttcctttcctactaactgaattagattagaaataagcagttaagtgagaaaaatagggaattagtgccagatatgttgaggtgtagtgttgacagggggtcttttaccttcagatgtttggtgcaacttagagaatgcagtggtagttgctggcccagcccatccaaacaacctcatttgtggggtgatacttcacagatgacttaaaataaatcccctgaaaataagaatctctggaagagatgacacgtgtatgtgcgtgtgtgcacgtgtgtgcgtgtgtgcacgtgtgtgtgtgtgtgtgttttggtggagtggtaggaggagggttggcttaatgatgagaatcattatttcttgaattggatgacactttccattcctgcaaagggagcgtgaggtccagccttgctaattcatgatcaaagtaatttgctgagagtagtggaaggaatgagttagcagaagatggcaaattgcattaatgagtagtaggaggaaaacaaataagcaaattttaatgtggttggaacatgttcctgagttgtatgaagggagacaggtaaattgagagtaatgttatgattgtttcatttgttggaaataaggaaaacttgcatggtttgaattgtggaacaataggtggtgtatgttaggagtcagaattatcatttaacatgtaagtttctgttttacttcagataaagaatgtcagctacctttcccatgttagcttctataccttttcttgaggtgaccataaataggtaatattgtctttcactgagattgttttaattagcgaagtgtgctgattaaatgtcttttcttgataaattattagaatgaataaaggatggtatttctagatagccttggttagtcagtgatttgtttaaatttagtgtggataagaatcacctgagggctttttaaacacagtttactgggtcctgaccttagtgtttctggcttattaggtataagtatgagaagttgatttttatcaagtttctagatgctacagatctaggaaccatacttagagtgctattatatgtcttttaatgtacactgatatggaaaaagcaatgatacatcatgttatcgcatattttaaaatgtgatcatgaccagggaatcttgaaagttgtcttatgtaataatgtaaatcaatgtagaataccatatatttttttcttgcaaggttattattaataggaatgactatttgtagccaatgtgttcaaatgcatgggaggacgtaaacacttcataatttcctggaaatacagacatatatcacttaaaataatacatttctaatataaatttaaattatctacgtgatatgaggaaacgagcttatttttgataattttcctagcagtgaatcaactatactcatttgaatctaaattaaattaaatttgagtaaattcagccatttctaaattcagtccatataaattcactttgggtaaaagaagacaaaattcctgaaaacagtagattaaaaaaataacattttaagagcaggtatgatctcaatgaatatagtgtggtaatttctgcctaatacctacccttttggaggaaggaaaggtaatcctgtttgtgtttaatgttaattactgaac

SEQ ID NO: 2 - DNA, 18 bp, synthetic construct
  misc_feature 1..18: note = PWAR5_primer F
  source 1..18: mol_type = other DNA
tgatgtgggtgttgatac

SEQ ID NO: 3 - DNA, 18 bp, synthetic construct
  misc_feature 1..18: note = PWAR5_primer R
  source 1..18: mol_type = other DNA
atcaagaacggaaactca

SEQ ID NO: 4 - DNA, 27 bp, synthetic construct
  misc_feature 1..27: note = qJFH1_primer F
  source 1..27: mol_type = other DNA
ttagtatgagtgtcgtacagcctccag

SEQ ID NO: 5 - DNA, 28 bp, synthetic construct
  misc_feature 1..28: note = qJFH1_primer R
  source 1..28: mol_type = other DNA
ggcatagagtgggtttatccaagaaagg

SEQ ID NO: 6 - DNA, 18 bp, synthetic construct
  misc_feature 1..18: note = HBV_primer F
  source 1..18: mol_type = other DNA
tcaccagcaccatgcaac

SEQ ID NO: 7 - DNA, 18 bp, synthetic construct
  misc_feature 1..18: note = HBV_primer R
  source 1..18: mol_type = other DNA
aagccacccaaggcacag

SEQ ID NO: 8 - DNA, 20 bp, synthetic construct
  misc_feature 1..20: note = GAPDH_primer F
  source 1..20: mol_type = other DNA
acagtcagccgcatcttctt

SEQ ID NO: 9 - DNA, 20 bp, synthetic construct
  misc_feature 1..20: note = GAPDH_primer R
  source 1..20: mol_type = other DNA
acgaccaaatccgttgactc